(19) United States
(12) Patent Application Publication
(10) Pub. No.: US 2011/0265237 A1
(43) Pub. Date: Nov. 3, 2011

(54) Eyewear Attachment System For A Helmet

(76) Inventors: Adam North Lazar, Williston, VT (US); James Patrick Radigan, Phoenix, AZ (US)

(21) Appl. No.: 13/082,245

(22) Filed: Apr. 7, 2011

Related U.S. Application Data
(60) Provisional application No. 61/321,536, filed on Apr. 7, 2010.

(51) Int. Cl. A61F 9/02; A42B 3/04

(52) U.S. Cl. 2/10

(57) ABSTRACT

An eyewear attachment system for a military helmet is provided having an auxiliary adapter with a width and a length spanning between a first end and a second end. The auxiliary adapter has an eyelet with an open center portion at the first end configured to receive an anchor member of a first connector therethrough to secure the auxiliary adapter to a helmet wherein the first connector is free to connect with a primary eyewear strap. The auxiliary adapter has an auxiliary connector at the second end configured to connect with a strap of a secondary eyewear, such as a goggle. The auxiliary adapter is pivotable about the anchor to accommodate a connection between the auxiliary adapter and a secondary eyewear strap.

This application claims the benefit of U.S. Provisional Patent Application No. 61/321,536, filed Apr. 7, 2010.

### TECHNICAL FIELD OF THE INVENTION

The present invention is directed to an attachment system for protective goggles, particularly an attachment system for mounting protective goggles to a protective helmet.

### BACKGROUND OF THE INVENTION

Protective helmets are used in various fields and worn by various users such as cyclists, motorcyclists, firefighters, skiers, military personnel including aircraft or helicopter pilots. Helmets generally include a rigid outer shell. The shell has a facial opening. The cavity within the helmet has protective and comfort padding members for contact with the user's head. The helmet is generally secured to the user's head by a flexible chin strap. Military and specifically aviator helmets often include a visor connected to the helmet and positionable in front of the user's eyes.

The present inventors have recognized a need for a helmet adapter that allows a pair of goggles and a visor to be attached to the helmet and used simultaneously without compromising or interfering with the use of the other component. The present inventors have recognized that a helmet user may need to use both a pair of goggles and a visor simultaneously. The present inventors have recognized the need for an adapter that connects to the helmet utilizing existing mounting holes presently available in the helmet. The present inventors recognized the need to utilize the mounting post and post hole of the male visor snap on a helmet to ensure that the helmet's structural integrity is not compromised.

### SUMMARY OF THE INVENTION

The invention comprises an eyewear attachment system for a helmet, having an auxiliary adapter with a width and a length spanning between a first end and a second end. The auxiliary adapter has an eyelet with an open center portion at the first end configured to receive an anchor member of a first connector therethrough to secure the auxiliary adapter to a helmet wherein the first connector is free to connect with a primary eyewear strap. The auxiliary adapter has an auxiliary connector at the second end configured to connect with a strap of a secondary eyewear, such as a goggle.

In another embodiment, the system comprises a goggle having a pair of straps for connecting opposite lateral sides of the goggle to the helmet, the goggle having a deployed position over the user's eyes and a standby position. The adapter has a deployed position and a standby position, the length of adapter directed forwardly toward a users eyes when in deployed position.

In another embodiment, the eyewear attachment system comprises a visor strap for connecting to a visor, the visor strap having a connector at first end opposite a second end, the second end configured to connect to the visor. The system also has a secondary eyewear strap for connecting to a secondary eyewear, the secondary eyewear strap having a connector at a first end opposite a second end, the second end configured to connect to the secondary eyewear, the secondary eyewear connector configured to connect to the auxiliary connector.

In one embodiment, the eyewear attachment system may have a strap system having an intermediate strap, a tri-glide strap adjuster, and an end strap portion. One end of the intermediate strap connects a goggle strap anchor to the tri-glide strap adjuster. The strap anchor connects to one side of a goggle. The opposite end of the intermediate strap connects to the tri-glide strap adjuster. The end strap portion has a female snap connector at a far end and the forward end is weaved through the tri-glide strap adjuster to secure the end strap portion to the tri-glide strap adjuster and provide user adjustability there through. The snap connector of the end strap portion is configured to connect to a male snap connector such as the one on the auxiliary adapter.

The auxiliary adapter allows a pair of goggles and a visor to be attached to a helmet and used simultaneously without compromising or interfering with the use of the other component. The auxiliary adapter connects to the helmet utilizing existing mounting holes presently available in the helmet. The auxiliary adapter allows numerous configurations of the visor and goggle with a helmet, such as, the goggles in a standby position on the helmet above the user's eyes while the visor is in a deployed position over the user's eyes, the goggles and visor both in the deployed position, the goggles and visor both in the standby position, and the goggles in the deployed position while the visor in the standby position. The auxiliary adapter allows—while the visor is attached—a secondary eyewear, such as goggles, to hang attached with only one strap to the helmet on one side so that the goggles are quickly deployable by a user by attaching the other strap to the opposite side of the helmet.

Numerous other advantages and features of the present invention will become readily apparent from the following detailed description of the invention and the embodiments thereof, and from the accompanying drawings.

### BRIEF DESCRIPTION OF THE DRAWINGS

FIG. 1 is a side perspective view of an auxiliary adapter used on a helmet with a visor in a standby position and goggles in a deployed position;

FIG. 2 is a side perspective view of the auxiliary adapter used on a helmet;

FIG. 3 is a side perspective view of the auxiliary adapter used on a helmet with goggles in the standby position;

FIG. 4 is a side perspective view of the auxiliary adapter used on a helmet with goggles in the deployed position;

FIG. 5 is a side perspective view of the auxiliary adapter used with a helmet where the visor is in the standby position;

FIG. 6 is a side perspective view of the auxiliary adapter used with a helmet where the visor is in the deployed position;

FIG. 7 is a side perspective view of an auxiliary adapter used on a helmet where the visor is in the standby position and the goggles are in a standby position over the visor;

FIG. 8 is a side perspective view of the auxiliary adapter used on a helmet where both the visor and goggles are in a deployed position;

FIG. 9 is a side perspective view of an auxiliary adapter used on a helmet where the visor is in the deployed position and goggles are in the standby position;

FIG. 10 is an opposite side perspective view of the auxiliary adapter used on a helmet where the visor is in the deployed position and the goggles are connected to one side of the helmet via the auxiliary adaptor with only one strap connected;

FIG. 11 is a bottom view of the auxiliary adapter;

FIG. 11A is a perspective view of the snap adapter and a portion of a helmet;

FIG. 12 is a side view of the tri-glide strap adjuster;

FIG. 13 is a side view of a portion of a strap system connected to a strap anchor with the tri-glide shown on the reverse side of that shown in FIG. 12;

FIG. 14 is a top view of a portion of the strap system connected to a strap anchor;

FIG. 15 is a side view of the end strap portion of the strap system; and

FIG. 16 is a schematic top view of the strap system connected to an anchor strap.

### DETAILED DESCRIPTION OF THE PREFERRED EMBODIMENTS

While this invention is susceptible of embodiment in many different forms, there are shown in the drawings, and will be described herein in detail, specific embodiments thereof with the understanding that the present disclosure is to be considered as an exemplification of the principles of the invention and is not intended to limit the invention to the specific embodiments illustrated.

The invention comprises a multiuse eyewear attachment system 150. In one embodiment, the multiuse system 150 comprises an auxiliary adapter 100. In another embodiment, the multi-use system comprises one or more goggle or secondary eyewear retaining straps 220, 230, and an auxiliary adapter 100 for connecting a secondary eyewear, such as a goggle 200, to the helmet while leaving a first male snap connector 60 available for connecting to a primary eyewear, such as a visor, to the helmet. In another embodiment, the multiuse system comprises the visor 70 with visor retaining straps 72, a secondary eyewear, such as the goggle 200 having goggle retaining straps 220, 230, and the auxiliary adapter 100 for securing the goggle retaining straps 220, 230 to the helmet while leaving the first male snap connector 60 available for connecting the visor retaining strap 72 to the helmet.

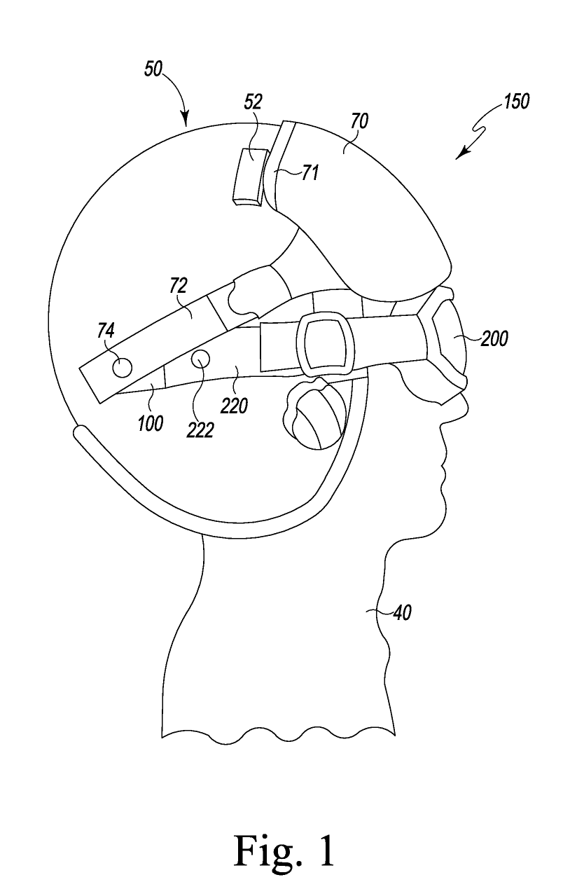

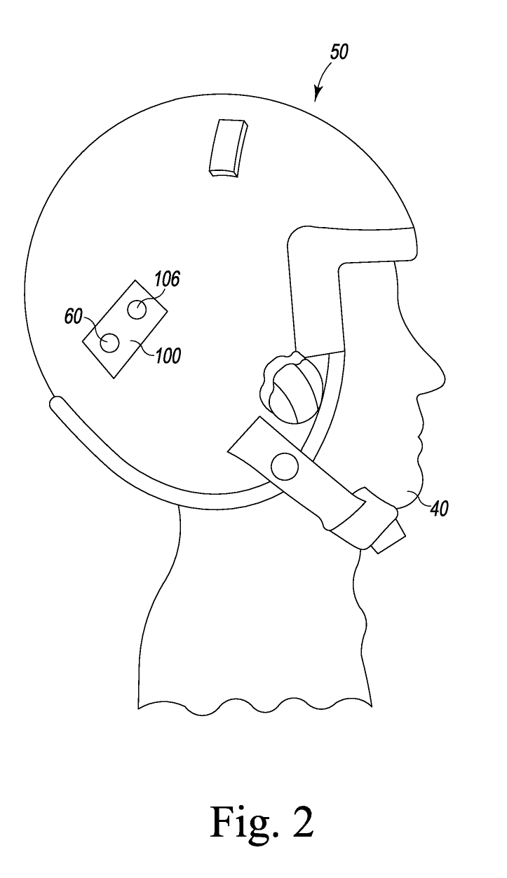

FIG. 1 shows a helmet 50 using the multiuse system 150 while being worn by a user 40. The helmet has a visor 70 that has a standby position and a deployed position. A visor stop 52 prevents further upward motion of the visor. A top portion 71 of the visor contacts the visor stop 52. The visor has a pair of visor straps 72, 76 the straps are is mirror image across a vertical center line of visor at the midpoint. The strap 72 has a visor strap female snap connector 74 at an end of the strap opposite the end of the strap that is connected to the visor. FIG. 1 shows that the snap connector 74 is connected to the male snap connector 60 (as shown in FIG. 2). Male snap connector 60 is anchored to the helmet. The helmet may provide a hole or a threaded hole to receive the male snap connector 60 and to secure it to the helmet.

The auxiliary adapter 100 is connected under the male snap connector 60. In one embodiment, the auxiliary adapter 100 is retained to the helmet by the male snap adapter. The auxiliary adapter 100 is pivotable about the connection to the helmet.

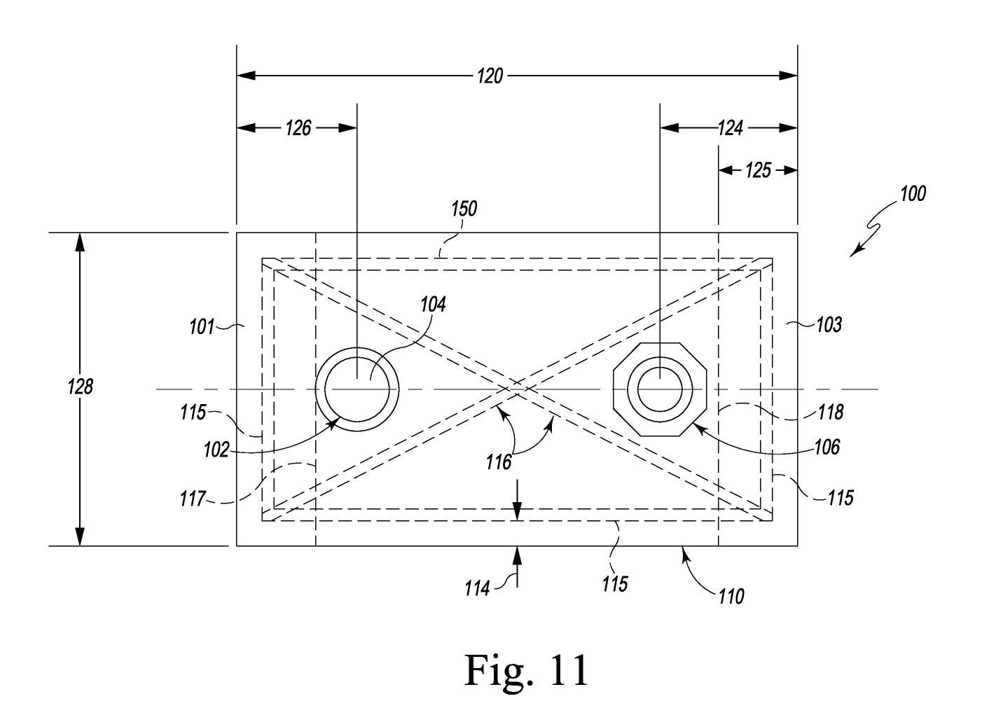

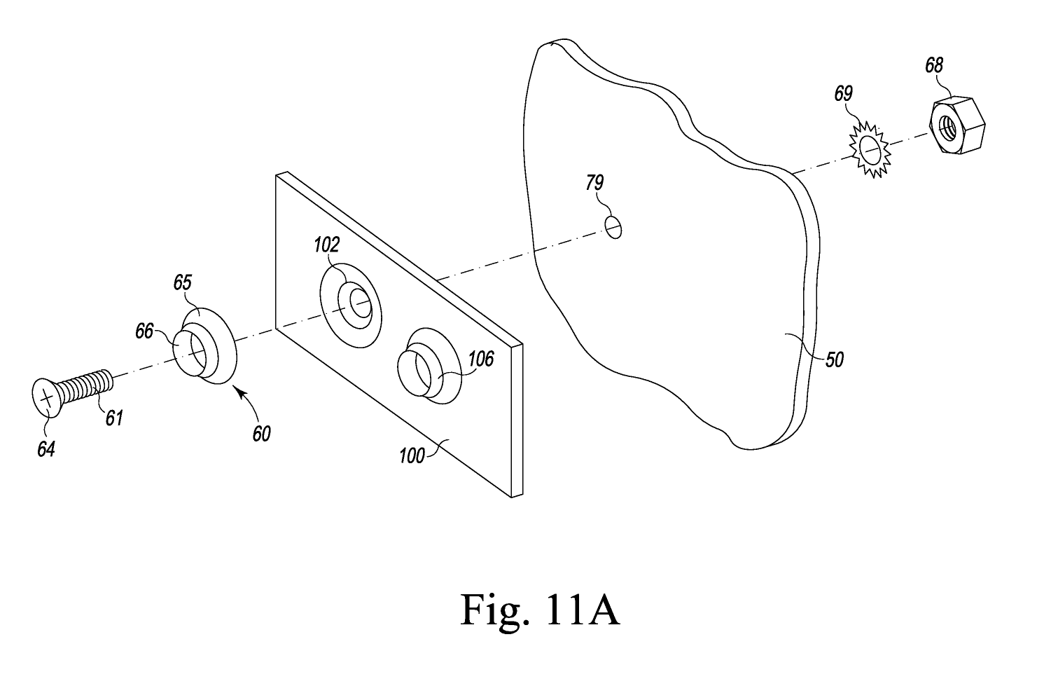

FIG. 11 shows a schematic view of the auxiliary adapter 100. Adapter 100 has a retained end 101 and an auxiliary attachment end 103. The retained end 101 has an eyelet 102 with an open center portion 104 for securing therethrough or thereon to a helmet. The auxiliary attachment end 103 has an auxiliary or second male snap connector 106 for connecting to a secondary or auxiliary eyewear, such as goggle 200. In one embodiment, adapter 100 is made out of material that may generally be used for straps such as canvas or nylon. The adapter may have diagonal stitching 116 running from one corner to a diagonally opposite corner to provide strength to the adapter. The adapter 100 may also have perimeter stitching 115 running along one or more edges of the adapter to provide strength and stability. The perimeter stitching 115 and the diagonal stitching 116 may be double or multiple rows of stitching such as shown in FIG. 11. The adapter may also have longitudinal end stitching 117, 118 that extends along the width of the adapter between the eyelet 102 and an outer edge of the retained end 101, or between the snap connector 106 and the outer edge of the auxiliary attachment end 103. The eyelet 102 may be located between converging diagonal stitching and perimeter stitching at the retained end 101. Likewise the snap connector 106 may be located between converging diagonal stitching and perimeter stitching at the auxiliary attachment end 103. The eyelet 102 may be located between converging diagonal stitching and longitudinal end stitching 117. The snap connector 106 may be located between converging diagonal stitching and longitudinal end stitching 118.

In one embodiment, the adapter 100 has the following measurements referring to FIG. 11, a width 128 of 40 mm and a length 120 of 70 mm. The adapter has a retained-edge-to-center-distance 126 between the longitudinal edge of the retained end and the center of the eyelet of 15 mm and an auxiliary-attachment-end-to-snap-connector distance 124 between the longitudinal edge of the auxiliary attachment end to the center of the snap connector of 17 mm. The adapter has an auxiliary-attachment-end-stitching-to-end-distance 125 between the longitudinal end stitching and the auxiliary attachment end 103 of 10 mm. The adapter has an eyelet 102 inside diameter of 5/16 of an inch. The adapter has a perimeter-to-stitching distance 114 from the edge to the perimeter stitching 3 mm. In one embodiment, the eyelet 102 is a brass grommet and the snap connector 106 is any male snap stud.

Returning to FIG. 1 and FIG. 11A, the male snap connector 60 is generally secured to the helmet 50 via an original helmet screw hole 79. The male snap connector maybe the original snap connector provided in the helmet at the screw hole 79. To install the adapter 100 on the helmet 50, the original screw (not shown) is removed and the snap connector 60 is freed from the helmet. The male snap connector 60 is placed over the eyelet 102 and a adapter screw or anchor member 64 is placed through the snap connector 60, through the eyelet 102, through at least a portion of the helmet screw hole 79 and is secured in place by a nut 68 on the inside of the helmet. The screw 64 and the nut create a friction fit pivot point about the screw to pivot the adapter 100. A lock washer 69 maybe placed between the inside of the helmet and the nut to provide a locking mechanism to prevent the screw from losing from its final toque setting. In another embodiment that hole 79 may be threaded so that the screw 64 engages the threads of the hole 79 rather than the nut 68. The screw may have a sloping head, which reduces toward the shaft portion 61 of the screw so as to conform is the inside of the raised portion 66 of the male snap connector 60. In one embodiment the auxiliary adapter 100 is pivotable about snap connector 60. The adapter screw may be longer than the original screw to account for the width of the adapter 100.

In FIG. 1, the auxiliary adapter 100 is secured under the snap connector 60. Snap connector 74 of the visor strap 72 is shown connected on the snap connector 60. The other end of the strap 72 extends upwardly relative to the goggle retaining strap 220 where the goggles are in a deployed position on the user. The visor strap overlays a portion of the auxiliary adapter 100, configured as shown in FIG. 1. The auxiliary adapter 100 extends from its retained end forward towards the users eyes and the male snap connector 106 is connected to a goggle female snap connector 222 to secure the goggle 200 in front of the user's eyes and to helmet via the auxiliary adapter.

FIG. 2 shows the auxiliary adapter secured to the helmet 50 by the snap connector 60 with the male snap connector 106 of the auxiliary adapter 100 visible. FIG. 2 shows the auxiliary adapter 100 connected to a helmet where the visor 70 and secondary eyewear such as goggle 200 are not connected or shown.

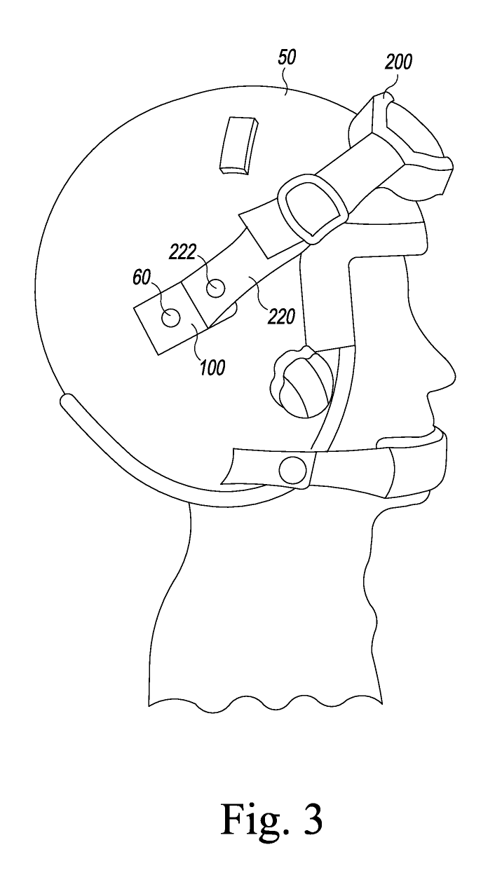

FIG. 3 shows the auxiliary adapter used with goggles in the standby position and where the visor 70 is not connected. The auxiliary adapter 100 is connected to the helmet at the snap connector 60. The snap connector 222 of the goggle strap 220 is connected to the male snap connector 106. The goggles are located on the helmet above the user's eyes in the standby position.

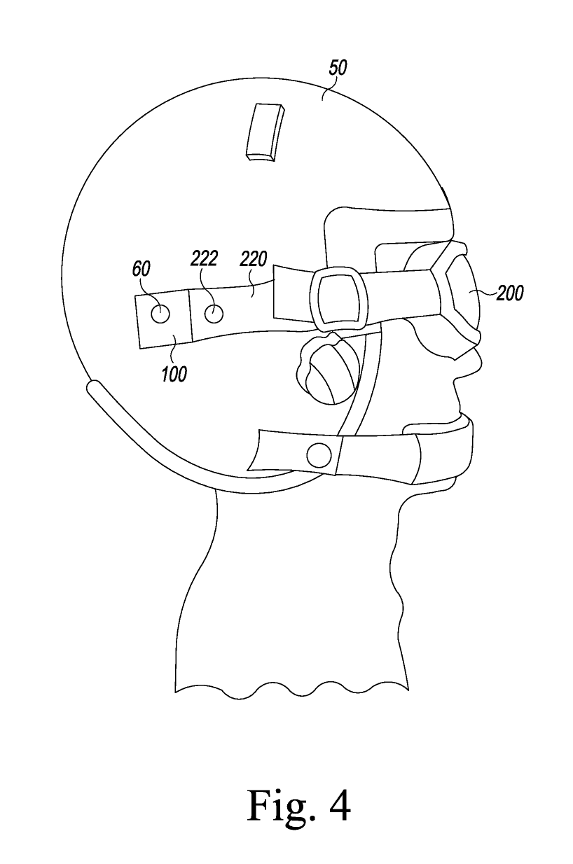

FIG. 4 shows the auxiliary adapter 100 connected to the helmet 50 at the snap connector 60. The goggles are in the deployed position over the user's eyes and connected to the male snap connector 106 by the goggle strap 220 and the visor is not connected.

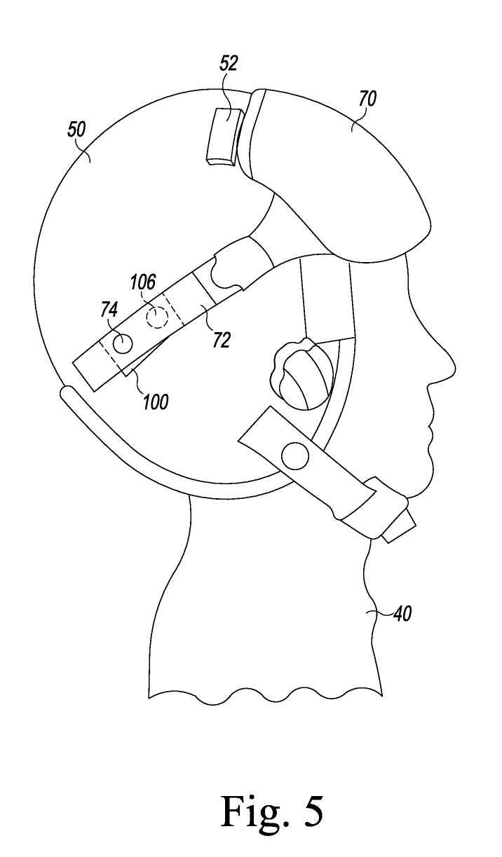

FIG. 5 shows the auxiliary adapter 100 connected to the helmet and positioned underneath a visor strap 72, which is attached to the male snap connector 60 at the visor strap connector 74. The visor is in the standby position. The auxiliary adapter 100 is shown by dashed lines underneath the visor strap 72 and includes the snap connector 106 also shown by dashed lines position underneath the visor strap 72. The goggles are not connected in FIG. 5.

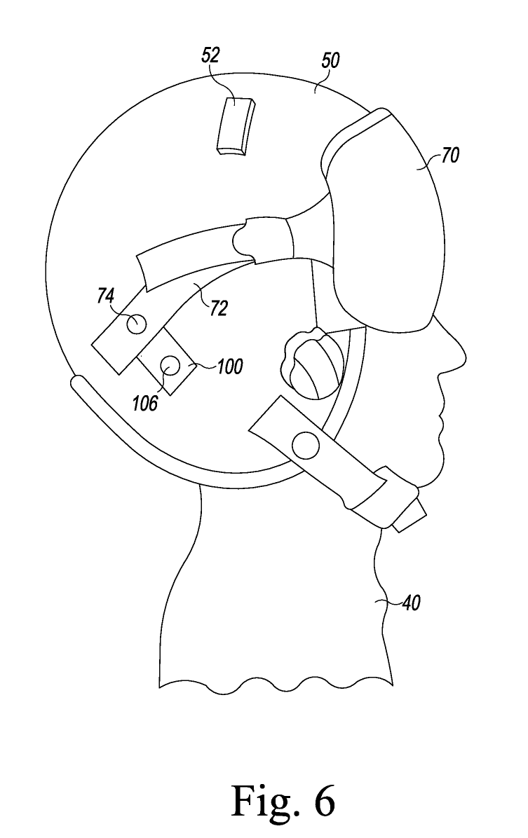

FIG. 6 shows the helmet with the visor 70 in the deployed position and the goggles are not connected. The visor is connected to the helmet at the snap connector 60. The auxiliary adapter 100 is shown connected to the helmet and ready to receive connection of a snap connector from a secondary eyewear, such as goggle 200.

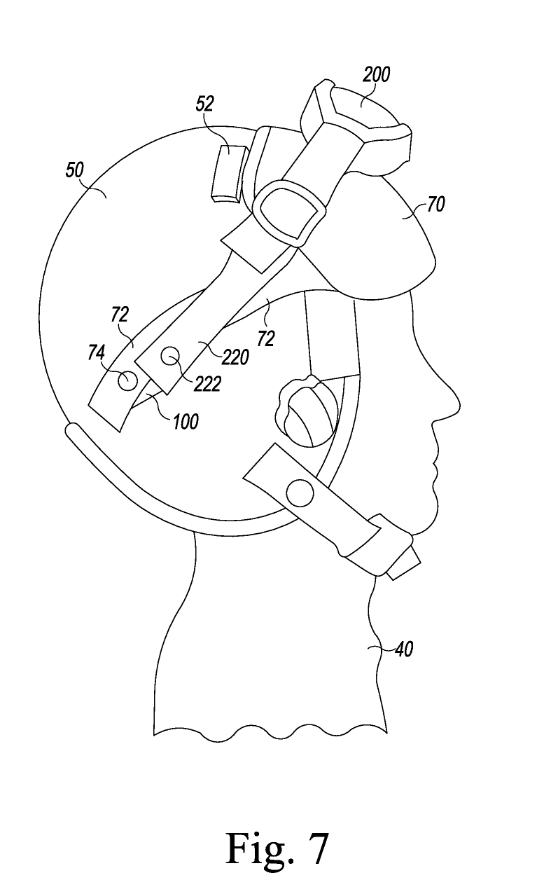

FIG. 7 shows a helmet with a visor 70 and a goggle 200 both in a standby position. The visor is connected to the helmet via the straps 72, 76 and the snap connectors 74, 78 at the snap connector 60. The visor strap connector 74 is connected to snap connector 60. The auxiliary adapter 100 is connected under snap connector 60 and extends outward from behind the visor strap 72 to connect with the goggle strap 220 at the snap connector 106. The goggle 200 are positioned in the standby position over the visor 70.

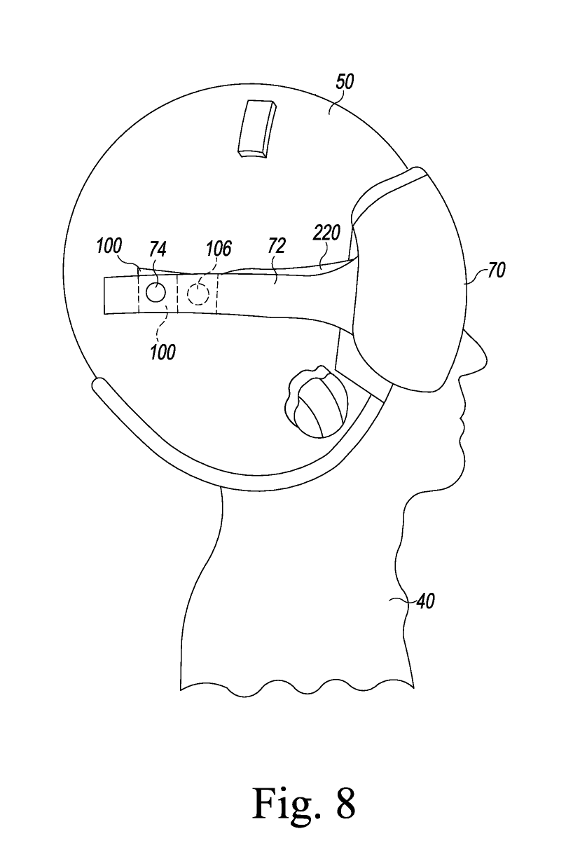

FIG. 8 shows the helmet 50 with a visor 70 and goggle 200 both attached and in the deployed position over the user's eyes. The visor strap 72 is connected to the snap connector 60 at connector 74. Underneath the visor strap 72 is the goggle strap 220 connected to adapter 100. Auxiliary adapter 100 is pivoted to extend substantially in parallel with the visor strap 72. The placement of the goggle 200 (not shown) in a deployed position over the user's eyes causes the strap 220 to extend in parallel under the visor strap 72 to connect to the auxiliary adapter 100 at a male snap connector 106. Depending on the location where the strap 72 attaches to the visor, or depending on the type of visor used, the distance of overlap of the visor strap and the goggle straps, as well as the angle of one strap to the other, may vary.

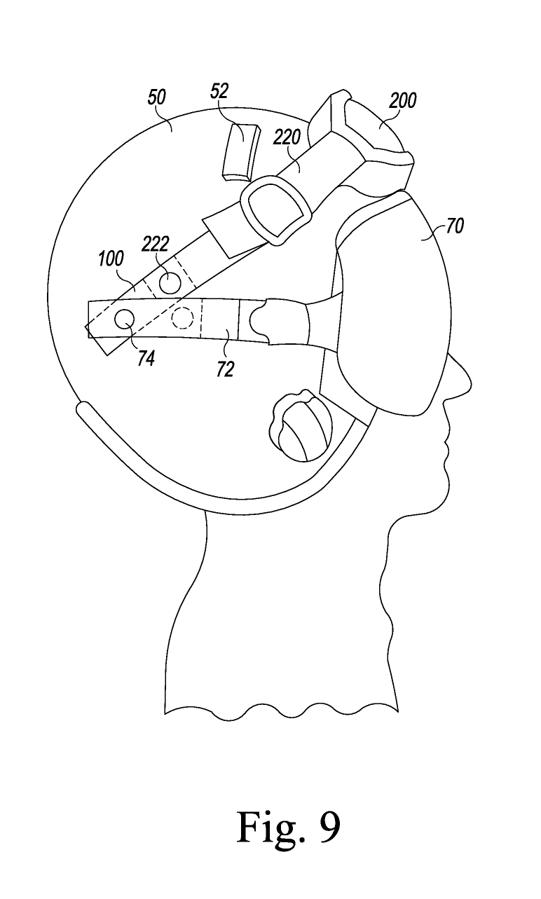

FIG. 9 shows the visor connected to the helmet in a deployed position and the goggle 200 in the standby position connected to the helmet 50 via the auxiliary adapter 100. The auxiliary adapter 100 is angled upward as drawn toward or positioned to accommodate connection with the goggle 200 in the standby position via strap 220.

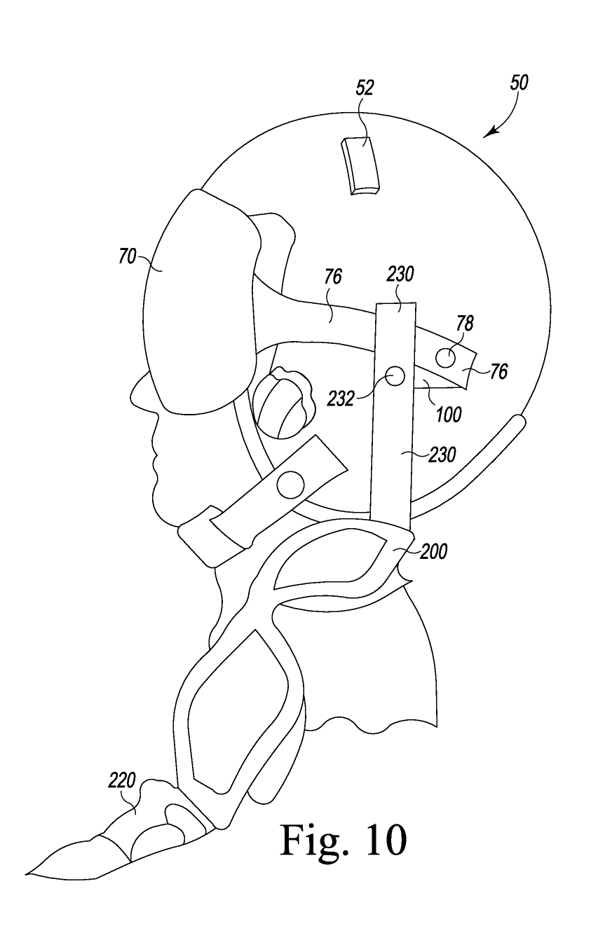

FIG. 10 shows the opposite side of the helmet as shown in FIG. 9. FIG. 10 shows the helmet 50 with the visor 70 in the deployed position and the goggle 200 with one strap 220 released from the auxiliary adapter 100 on the opposite side and the other strap 230 of the goggles connected to the auxiliary adapter 100. The auxiliary adapter 100 is positioned under the visor strap 76 at a rear portion of the helmet. The auxiliary adapter may extend upward towards a top edge of visor strap 76 or may extend under and below the visor strap 76. In one embodiment, the auxiliary adapter 100 is twisted over the top edge by the strap 76 so that a snap connector 232 of the goggle strap 230 is positioned upward and connects to the auxiliary adapter.

In one embodiment, the goggle 200 is the goggle labeled 190 or 300 in U.S. Pat. No. 7,648,233, each of which is herein incorporated by reference to the extent not inconsistent with the present disclosure. In one embodiment, the goggle 200 has strap anchors 300, 310 as described in U.S. Pat. No. 7,648,233, which is herein incorporated by reference.

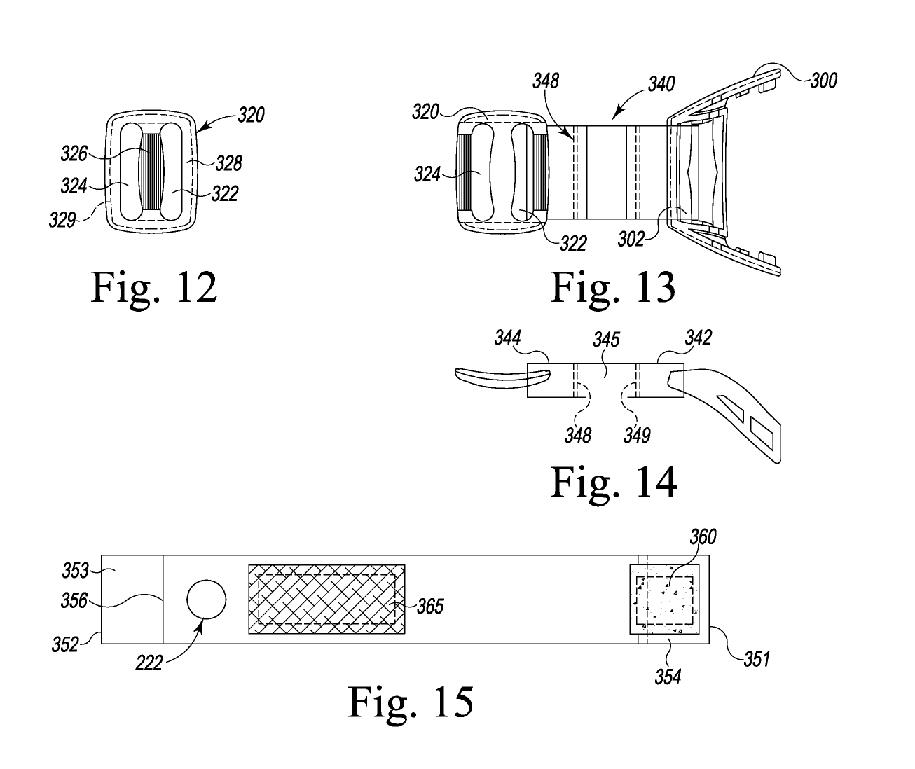

Referring to FIGS. 13-16, the strap 220 may comprise a strap system 301 including an intermediate strap 340, a tri-glide strap adjuster 320, and an end strap 350. A left side strap anchor 300 is shown in FIG. 13. The intermediate strap 340 connects the strap anchor 300 to the tri-glide strap adjuster 320 at one end of the strap 340 at an intermediate connecting slot 322 and also at an opposite end of the strap 340 to an intermediate strap connecting slot 302. The intermediate strap 340 has a goggle loop 342, and a triglide loop 344 formed by stitching 349, 348 and spaced apart by an intermediate strap middle portion 345.

FIG. 15 shows the end strap 350. The end strap 350 has a first end 351 and a second end 352. The first end 351 has a first end loop 354 and the second end 352 has a second end loop 353 each defined in part by a stitching 357, 356 respectively. The first end has an outward facing side as shown in FIG. 15 that has a hook and loop fastener component 360, 365. The hook portion 360 is located at the first end 351 and a loop portion 365 is located near the second end 352. A snap connector 222 is located between the loop portion 365 and the second end 352.

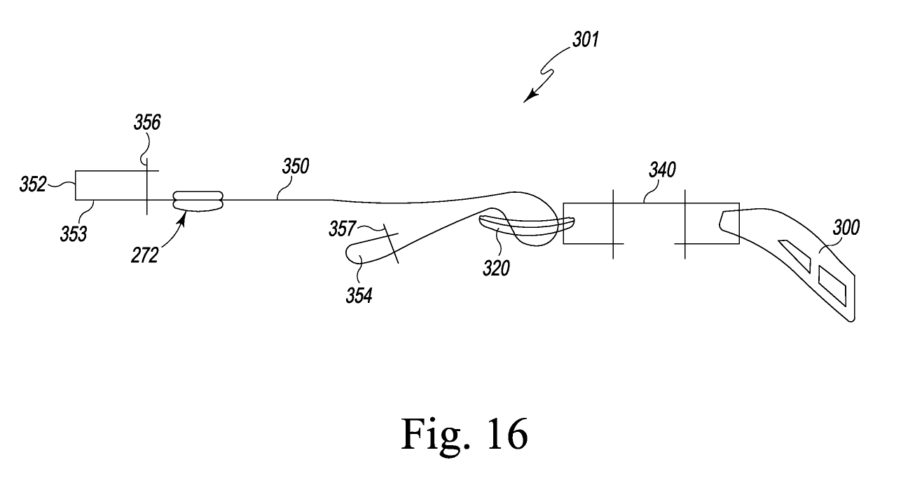

FIG. 16 shows the strap system 301. The strap anchor 300 is connected to the intermediate strap 340 at one end and at an opposite end of the intermediate strap 340 to tri-glide strap adjustor 320 is connected. The first end 351 of the end strap 350 is weaved through the tri-glide strap adjustor 320, up through a first slot 322, over a middle portion 326 down through a second slot 324 and out between adjustor 320 and a portion of the end strap 350. The hook portion 360 is configured to connect to the loop portion 365 to prevent the first end 351 from hanging loose. Snap connector 222 is configured to connect to auxiliary adapter 100 as explained above to secure a goggle to the helmet.

While connectors 60, 74, 106, 222, 232 are described as snap type connectors, one skilled in the art will recognized that other fasteners suitable for connecting straps may also be used, such as hook and loop fasteners and anchor-slot connectors. While various snap connections have been described as male or female snap connections, one skilled in the art will recognize that the male and female connections for a pair of snap connections may be swapped from that which is described, so that the side of the connection described as having a male connection has a female connection and the side of the connection having a female connection has a male connection. Moreover, the adapter 100 and the strap system 301 may be made of flame-resistant material.

From the foregoing, it will be observed that numerous variations and modifications may be effected without departing from the spirit and scope of the invention. It is to be understood that no limitation with respect to the specific apparatus illustrated herein is intended or should be inferred.

The invention claimed is:

1. An eyewear attachment system for a military helmet, comprising:
an auxiliary adapter having a width and a length spanning between a first end and a second end;
an eyelet with an open center portion at the first end configured to receive an anchor member connected to a first connector therethrough to secure the auxiliary adapter to the military helmet wherein the first connector is free to connect with a primary eyewear strap;
an auxiliary connector at the second end configured to connect with a strap of a secondary eyewear; and
the auxiliary adapter pivotable about the anchor member to accommodate a position of the secondary eyewear.

2. The eyewear attachment system of claim 1, wherein the auxiliary adapter has a deployed position and a standby position, the length of the auxiliary adapter extends forwardly toward a users eyes when in the deployed position.

3. The eyewear attachment system of claim 1, wherein the secondary eyewear is a goggle.

4. The eyewear attachment system of claim 1, comprising a first connector and a primary eyewear having at least one strap connectable to the first connector.

5. The eyewear attachment system of claim 1, wherein the auxiliary adapter pivotable 180 degrees about the connection to the helmet.

6. The eyewear attachment system of claim 1, wherein the secondary eyewear strap comprises a strap system having an intermediate strap capable of connecting to the secondary eyewear, an end strap configured to connect to the auxiliary connector, and a strap adjuster configured to connect the intermediate strap to the end strap.

7. An eyewear attachment system for a helmet, comprising:
at least two auxiliary adapters each having a width and a length spanning between a first end and a second end, the length being greater than the width;
each auxiliary adapter having a first connector at the first end configured to be anchored to a side of the helmet and connectable to a first eyewear strap of a first eyewear, and an auxiliary connector at the second end configured to connect with a secondary eyewear strap of a secondary eyewear; and
each auxiliary adapter pivotable at the first connector to accommodate a position of the secondary eyewear.

8. The eyewear attachment system of claim 7, comprising a goggle having a pair of straps for connecting opposite lateral sides of the goggle to the helmet, the goggle positionable in a deployed position over a user's eyes, each strap having goggle strap connector located adjacent an end of the strap opposite the goggle, each goggle strap connector configured to connect to one of the auxiliary connectors.

9. The eyewear attachment system of claim 7, wherein the first connector and the auxiliary connector are male snap connectors.

10. The eyewear attachment system of claim 7, wherein the first end has a first edge and the second end has a second edge, the first connector is closer to the first edge than to the auxiliary connector, and the auxiliary connector is closer to the second edge than to the first connector.

11. The eyewear attachment system of claim 7, wherein each auxiliary adapter has double rowed strength-enhancing peripheral edge stitching and a set of crossing double rowed strength-enhancing diagonal stitching.

12. The eyewear attachment system of claim 7, comprising a pair of visor straps for connecting to a visor, each visor strap having a visor strap connector at a first visor strap end opposite a second visor strap end, the second visor strap end configured to connect to the visor, the visor strap connector connectable to one of the first connectors.

13. The eyewear attachment system of claim 7, wherein each auxiliary adapter has an eyelet with an open center portion at the first end configured to receive an anchor member of the first connector therethrough to secure the auxiliary adapter to the helmet wherein the first connector is capable of connecting to a primary eyewear strap.

14. A method of using more than one eyewear with a helmet simultaneously, comprising the steps of:
connecting a first side auxiliary adapter to a first side of the helmet at a first primary eyewear connector while leaving the first primary eyewear connector free to connect with a strap of a primary eyewear;
connecting a second side auxiliary adapter to an opposite second side of the helmet at a second primary eyewear connector while leaving the second primary eyewear connector free to connect with a second strap of the primary eyewear;
connecting a first secondary eyewear strap of a secondary eyewear to a first side auxiliary connector on the first side auxiliary adapter;
connecting a second secondary eyewear strap of the secondary eyewear to a second side auxiliary connector on the second side auxiliary adapter;
pivotally connecting a first primary eyewear strap to the first primary eyewear connector; and
pivotally connecting a second primary eyewear strap to the second primary eyewear connector.

15. The method of claim 14, wherein the step of pivotally connecting a first primary eyewear strap comprises the step of pivotally connecting a first primary eyewear strap to the first primary eyewear connector so that a portion of the first primary eyewear strap overlies a portion the first side auxiliary adapter; and
wherein step of pivotally connecting a second primary eyewear strap comprises the step of pivotally connecting a second primary eyewear strap to the second primary eyewear connector so that a portion of the second primary eyewear strap overlies a portion the second side auxiliary adapter, wherein the primary eyewear is movable between a standby position above a user's eyes and a deployed position over the user's eyes.

16. The method of claim 14, wherein the step of connecting a first side auxiliary adapter comprises the step of pivotally connecting a first side auxiliary adapter to a first side of a helmet; and wherein the step of connecting a second side auxiliary adapter comprises the step of pivotally connecting the second side auxiliary adapter to an opposite second side of the helmet.

17. The method of claim 14, wherein the step of connecting a first auxiliary adapter to a first side of a helmet comprises the steps of,
placing the first auxiliary adapter between the primary connector and the helmet; and,
connecting the first auxiliary adapter to the helmet by securing the first primary connector to the helmet through the first side auxiliary adapter.

18. The method of claim 17, wherein the step of connecting a first auxiliary adapter to a first side of a helmet comprises the step of, before placing the first auxiliary adapter, removing the first primary connector from the helmet.

19. An eyewear attachment adapter configured to enable the simultaneous use of more than one eyewear with a helmet, comprising:
an auxiliary strap having a width, and a length spanning between a first end and a second end;
a first connector area at the first end capable of being secured to a side of a helmet;
an auxiliary connector at the second end configured to connect with a secondary eyewear strap of a secondary eyewear.

20. An eyewear attachment adapter of claim 19, wherein the first connector area comprises an eyelet with an open center portion at the first end configured to receive an anchor member connected to a first connector to secure the auxiliary strap to a helmet wherein the first connector is free to connect with a primary eyewear strap.

21. An eyewear attachment adapter of claim 20, wherein the auxiliary strap is pivotable about the anchor member when secured to the helmet.
